(19) United States
(12) Patent Application Publication
(10) Pub. No.: US 2026/0069313 A1
(43) Pub. Date: Mar. 12, 2026

(54) INSERTION TOOL FOR INSERTING AT LEAST ONE MEDICAL DEVICE INTO A BODY TISSUE

(71) Applicant: Roche Diabetes Care, Inc., Indianapolis, IN (US)

(72) Inventor: Friedhelm Kreutz, Nieder-Olm (DE)

(21) Appl. No.: 19/389,959

(22) Filed: Nov. 14, 2025

Related U.S. Application Data
(63) Continuation of application No. PCT/EP2024/063325, filed on May 15, 2024.

(30) Foreign Application Priority Data
May 17, 2023 (EP) 23 173 964.0

(51) Int. Cl. A61B 17/34; A61B 5/145; A61B 17/00; A61B 50/22

(52) U.S. Cl. CPC A61B 17/3468; A61B 5/14503; A61B 50/22; A61B 2017/00486; A61B 2017/00526; A61B 2560/063

(57) ABSTRACT

An insertion tool for inserting at least one insertable part of a medical device into a body tissue of a subject is proposed. The insertion tool comprises a shaft configured for receiving the medical device. The shaft comprises an insertable first end and an adjustable second end.

### RELATED APPLICATIONS

This application is a continuation of International Application No. PCT/EP2024/063325, filed May 15, 2024, which claims priority to EP 23 173 964.0, filed May 17, 2023, the entire disclosures of both of which are hereby incorporated herein by reference.

### BACKGROUND

This disclosure relates to an insertion tool for inserting at least one medical device into a body tissue, a kit, an adapter, a needle comb and a cutting tool. The medical device may specifically be configured for detecting at least one analyte in a body fluid of a subject. The devices may be applied in the field of continuous monitoring of the analyte in the body fluid of the subject, specifically in the field of home care and in the field of professional care, such as in hospitals. Other applications, however, are also feasible.

Monitoring certain body functions, more particularly, monitoring one or more analyte concentrations such as one or more metabolite concentrations in a body fluid of a subject, plays an important role in the prevention and treatment of various diseases. Such analytes can include by way of example, but not exclusively, glucose, lactate, cholesterol or other types of analytes and metabolites. Without restricting further possible applications, this disclosure will be described in the following text with reference to glucose monitoring. However, additionally or alternatively, this disclosure can also be applied to other types of analytes.

Blood glucose monitoring, besides by using optical measurements, specifically may be performed by using electrochemical biosensors. In addition to so-called spot measurements, in which a sample of a body fluid is taken from a user in a targeted fashion and examined with respect to the analyte concentration, continuous measurements are increasingly becoming established. Thus, in the recent past, continuous measuring of glucose in the interstitial tissue (also referred to as continuous monitoring, CM), for example, has been established as another important method for managing, monitoring and controlling a diabetes state.

In the process, an active sensor region is applied directly to a measurement site, which is generally arranged in an interstitial tissue, and, for example, converts glucose into electrical charge by using an enzyme (e.g., glucose oxidase, GOD), which charge is related to the glucose concentration and can be used as a measurement variable. Examples of such transcutaneous measurement systems are described in U.S. Pat. No. 6,360,888 B1 or in U.S. Publication No. 2008/0242962 A1. Hence, current continuous monitoring systems typically are transcutaneous systems or subcutaneous systems. This means that the medical device, in this case the analyte sensor or at least a measuring portion of the sensor, is arranged by using an insertion system under the skin of the subject.

Usually, a patch is applied to the upper arm for continuous measurement of the blood glucose level. An evaluation and control part of the insertion system (also referred to as a patch) is generally situated outside of the body of the subject, e.g., outside of the human or animal body. In the process, the medical device is generally applied using an insertion instrument, which is likewise described in U.S. Pat. No. 6,360,888 B1 in an exemplary fashion. Other types of insertion instruments are also known. Usually, a subcutaneous analyte sensor is based on a plastic substrate and, thus, cannot be inserted by itself and demands a metallic cannula, denoted as insertion cannula. The insertion cannula typically possesses a special void for placing the medical device in it. During the insertion process, the insertion cannula containing the medical device, penetrates the skin and retracts leaving the medical device, e.g., the analyte sensor, inserted. An insertion depth is a decisive factor in ensuring the safe function of the medical device. At the same time, a minimum puncture depth is aimed for in order to reduce bleeding and pain. In known insertion systems, the insertion cannula is glued to a needle head using a fix mechanical stop. In case medical studies would show that a length if the insertion cannula is to long (e.g., that a shorter insertion needle would reduce bleeding and pain during application and carrying), this would have significant influences on a production of the insertion cannula. Specifically, amended production tools for production of the insertion cannula have long delivery times, e.g., of months or even a year, such that even a delay for a whole project can occur, e.g., if it becomes apparent only after completion of the studies that a shorter needle is necessary.

It is known, e.g., from CN 103328022 B to adjust an insertion depth of an insertion tool such as a needle via an insertion mechanism of an insertion device.

### SUMMARY

This disclosure teaches an insertion tool for inserting at least one medical device into a body tissue, a kit, an adapter, a needle comb and a cutting tool which at least partially address the above-mentioned technical challenges. Specifically, easy and quick adjustment of a length of the insertion cannula can be achieved without the necessity of redesigning any production tools, in particular, a cutting tool.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “insertable part,” “adjustable portion,” and “undercut,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities.

Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, an insertion tool for inserting at least an insertable part of a medical device into a body tissue of a subject is disclosed.

The insertion tool comprises a shaft configured for receiving the medical device. The shaft comprises an insertable first end and an adjustable second end.

This disclosure teaches allowing for an adjustment of a length of the insertion tool after a punching step. The adjustment can be performed without adapting a cutting tool or the like such as without the necessity of redesigning any tools, in particular, a cutting tool and/or to change of any interfaces with further elements of an insertion system for inserting at least an insertable part of a medical device into a body tissue of a subject.

Therefore a requalification of the cutting tool is not needed. An adaption of the production can be realized easily and quickly. Therewith a length adaption may be realized in a time and cost efficient manner.

The term “subject” as used herein is a broad term and is to be given its ordinary and customary meaning to a person of ordinary skill in the art and is not to be limited to a special or customized meaning. The term specifically relates to a human being or an animal, independent from the fact that the human being or animal, respectively, may be in a healthy condition or may suffer from one or more diseases. The subject may be a patient. As an example, the subject may be a human being or an animal suffering from diabetes.

The subject may be a user, e.g., a patient, intending to monitor an analyte value, such as a glucose value, in the user's body tissue and/or to deliver medication, such as insulin, into the user's body tissue. However, in an embodiment, the user may be different from the subject. Additionally or alternatively, this disclosure may be applied to other types of users or patients.

The term “medical device” (or “medical instrument”) as used herein is a broad term and is to be given its ordinary and customary meaning to a person of ordinary skill in the art and is not to be limited to a special or customized meaning. The term specifically may refer, without limitation, to an arbitrary element or article being configured for use in the field of medical technology, exemplarily in the field of medical analytics or medical diagnostics. The medical device may be configured for performing at least one medical function and/or for being used in at least one medical process, such as one or more of a therapeutic process, a diagnostic process or another medical process.

The medical device may be configured to be mounted on a skin site, such as a skin site of an extremity of the subject. The extremity may be selected from the group consisting of: an arm, exemplarily an upper arm; a stomach; a shoulder; a back; hip; a leg. Exemplarily, the extremity may be the upper arm. However, also other applications may be feasible.

The medical device may comprise at least one component which may be configured to stay outside of the body tissue. Further, the medical device may comprise at least one insertable portion. The insertable portion may be configured for being inserted into the body tissue of the subject. Exemplarily, the insertion tool may be configured for inserting the insertable portion of the medical device into the body tissue of the subject.

The medical device is selected from the group consisting of: a biosensor, specifically an analyte sensor for detecting at least one analyte in a bodily fluid; an infusion device, e.g., a drug infusion device configured for infusing a liquid drug such as insulin or any other liquid drug into a tissue of the subject; and an electrical stimulating device, e.g., configured for stimulating a body tissue of the subject. Such infusion devices include infusion pumps that can be attached to the skin of a patient also known as patch pumps or infusion sets that are connected to an infusion pump often worn in the pocket of a patient and connected to the infusion set by a tubing to deliver the drug from the pump into the skin. Known patch pumps include insulin pumps such as Roche's Accu-Chek® Solo Micropump or Terumo's Medisafe patch pump. Known infusion sets, in turn, include Roche's Accu-Chek® Insight Flex or Tandem's Diabetes AutoSoft Infusion set or the infusion sets manufactured by Convatec® such as Neria Guard.

For example, the medical device may comprise at least one analyte sensor device. The analyte sensor device may comprise at least one analyte sensor configured for detecting at least one analyte in a body fluid of the subject. The insertion tool may be configured for inserting at least a part of the analyte sensor into the body tissue of the subject, e.g., at least one insertable portion.

The term “analyte sensor device” or simply “analyte sensor” as used herein is a broad term and is to be given its ordinary and customary meaning to a person of ordinary skill in the art and is not to be limited to a special or customized meaning. The term specifically may refer, without limitation, to a device configured for detecting at least one analyte in a body fluid of the subject.

The term “analyte” as used herein is a broad term and is to be given its ordinary and customary meaning to a person of ordinary skill in the art and is not to be limited to a special or customized meaning. The term specifically may refer, without limitation, to a chemical and/or biological substance which takes part in the metabolism of the body of the subject. Exemplarily, the analyte may be a metabolite or a combination of two or more metabolites. As an example, the analyte may be selected from the group consisting of: glucose, lactate, triglycerides, cholesterol. A preferred analyte is glucose. Still, other analytes or combinations of two or more analytes may be detected. The body tissue exemplarily may be or may comprise fatty tissue and/or interstitium. Other types of body tissue, however, are feasible.

The analyte sensor may be configured for being used in qualitatively and/or quantitatively detecting the at least one analyte. The term “analyte sensor” as used herein is a broad term and is to be given its ordinary and customary meaning to a person of ordinary skill in the art and is not to be limited to a special or customized meaning. The term specifically may refer, without limitation, to a sensor which is capable of qualitatively or quantitatively detecting the presence and/or the concentration of the at least one analyte.

The analyte sensor may be an electrochemical analyte sensor. The analyte sensor may comprise at least two electrodes. Specifically, the analyte sensor may comprise at least one two-electrode sensor. The two-electrode sensor may comprise precisely two electrodes, such as a working electrode and at least one further electrode such as a counter electrode, e.g., a working electrode and a combined counter/reference electrode. The working electrode may comprise a working electrode pad and, optionally, at least one test chemical disposed thereon. The counter electrode may comprise a counter electrode pad. Additionally and optionally, one or more redox materials may be disposed thereon. The analyte sensor may further comprise one or more leads for electrically contacting the electrodes. The leads may, during insertion or at a later point in time, be connected to one or more electronic components. For example, the leads may already be connected to the electronic components before insertion of the analyte sensor. For example, the analyte sensor may be a needle-shaped or a strip-shaped analyte sensor having a flexible substrate and the electrodes disposed thereon. As an example, the analyte sensor may have a total length of 5 mm to 50 mm, specifically a total length of 7 mm to 30 mm. The term “total length” within the context of this disclosure relates to the overall length of the analyte sensor which means a portion of the analyte sensor which is inserted and the portion of the analyte sensor which may stay outside of the body tissue. The portion of the analyte sensor which is inserted is also called the in vivo portion, the portion of the analyte sensor which may stay outside of the body tissue is also called the ex vivo portion. For example, the in vivo portion has a length in the range from 3 mm to 12 mm. The analyte sensor may further comprise a biocompatible cover, such as a biocompatible membrane which fully or partially covers the analyte sensor and which prevents the test chemical from migrating into the body tissue and which allows for a diffusion of the body fluid and/or the analyte to the electrodes. Other embodiments of electrochemical analyte sensors, such as three-electrode sensors, may be feasible. For example, the three-electrode sensor may comprise, in addition to the working electrode and the counter electrode, a reference electrode. Such analyte sensors are generally known in the art and include continuous glucose sensor systems such as Dexcom's G6 or G7 glucose sensor system, Medtronic's Enlite glucose sensor or Abbott's Freestyle Libre 2 or 3.

The analyte sensor may be an optical analyte sensor. For example, the analyte sensor may comprise a flexible light guide with glucose sensitive coating at its end and/or a tube like carrier with functional elements at inner or outer walls. Other embodiments of the analyte sensor may be possible too.

The term “bodily fluid,” also referred to interchangeably herein as “body fluid,” as used herein is a broad term and is to be given its ordinary and customary meaning to a person of ordinary skill in the art and is not to be limited to a special or customized meaning. The term specifically relates to an arbitrary fluid which typically is present in a body or body tissue of a user or a patient and/or which may be produced by the body of the user or the patient. As an example for body tissue, interstitial tissue may be named. Thus, as an example, the bodily fluid may be selected from the group consisting of blood and interstitial fluid. However, additionally or alternatively, one or more other types of body fluids may be used, such as saliva, tear fluid, urine or other body fluids. During detection of at least one analyte, the body fluid may be present within the body or body tissue.

The medical device may comprise at least one electronics unit. The analyte sensor may be operably connected to the electronics unit. The term “electronics unit” as used herein is a broad term and is to be given its ordinary and customary meaning to a person of ordinary skill in the art and is not to be limited to a special or customized meaning. The term specifically may refer, without limitation, to an arbitrary unit, such as a unit which may be handled as a single piece, which is configured for performing at least one electronic function. For example, in case the medical device is the medical device for detecting the analyte in the body fluid of the user, the electronics unit may have at least one interface for being connected to the analyte sensor, wherein the electronics unit may provide at least one electronic function interacting with the analyte sensor, such as at least one measurement function. The electronics unit may be configured for one or more of determining and/or controlling a detection of the analyte and/or transmitting measurement data to another component. Specifically, the electronics unit may be configured for one or more of performing a measurement with the analyte sensor, performing a voltage measurement, performing a current measurement, recording sensor signals, storing measurement signals and/or measurement data, transmitting sensor signals to another component. Thus, the electronics unit specifically may comprise at least one of: a voltmeter, an ammeter, a potentiostat, a voltage source, a current source, a signal receiver, a signal transmitter, an analog-digital converter, an electronic filter, a data storage device, an energy storage. For example, the electronics unit may be embodied as a transmitter or may comprise at least one transmitter configured for transmitting data to a remote computer or to a remote device.

The medical device further may comprise at least one electronic remote device configured to communicate and/or control with the medical device. The electronic remote device may be selected from a personal computer, a wearable, a smartphone, a proprietary remote control, a tablet or a server.

For example, the medical device may comprise at least one infusion device. The term “infusion device” as used herein is a broad term and is to be given its ordinary and customary meaning to a person of ordinary skill in the art and is not to be limited to a special or customized meaning. The term specifically relates to a device configured for delivering and/or infusing at least one medication into the body tissue of the user, e.g., for delivering and/or infusing insulin into the body tissue of the user. The infusion device may be a drug infusion device. The infusion device may comprise at least one infusion cannula. The insertion tool may be configured for inserting at least a part of the infusion cannula into the body tissue of the subject. The term “infusion cannula” as used herein is a broad term and is to be given its ordinary and customary meaning to a person of ordinary skill in the art and is not to be limited to a special or customized meaning. The term exemplarily may refer, without limitation, to a hollow tube configured for delivering and/or infusing a medication into the body tissue of the subject, e.g., for delivering and/or infusing insulin into the body tissue of the subject.

For example, the medical device may comprise at least one electrical stimulation device. The term “electrical stimulation device” as used herein is a broad term and is to be given its ordinary and customary meaning to a person of ordinary skill in the art and is not to be limited to a special or customized meaning. The term specifically relates to a device configured for applying at least one electrical stimulation to the body tissue. The electrical stimulation device may comprise a stimulation electrode. The insertion tool may be configured for inserting at least a part of the stimulation electrode into the body tissue of the subject.

The term “inserting” as used herein is a broad term and is to be given its ordinary and customary meaning to a person of ordinary skill in the art and is not to be limited to a special or customized meaning. The term specifically may refer, without limitation, to an action or process of one or more of transcutaneously or subcutaneously implanting and/or positioning the medical device, exemplarily the insertable portion of the medical device, into the body tissue of the subject. The medical device may partially be inserted into the body tissue. The insertion of the medical device may be performed by using the insertion tool. After insertion, at least an insertable part of the medical device may remain in the body tissue of the subject for a predetermined period of time, such as for several hours, exemplarily for one or more days, such as for up to one week, or such as for up to two weeks or even more.

The term “insertion tool” as used herein is a broad term and is to be given its ordinary and customary meaning to a person of ordinary skill in the art and is not to be limited to a special or customized meaning. The term specifically relates to an arbitrary element which may be insertable at least partially into the body tissue, particularly in order to deliver or to transfer a further element. The insertion tool may be configured for supporting the insertion of the insertable part of the medical device. The insertion tool may be configured for transcutaneously or subcutaneously inserting at least the insertable part of the medical device into the body tissue, such as by performing an incision or a puncture in a skin of the subject and by transferring the medical device partially into the body tissue. The insertion tool may be removed fully or partially after inserting the medical device at least partially into the body tissue of the subject. Exemplarily, the insertion tool may be configured for supporting the insertion of the insertable portion of the medical device. The insertion tool may comprise a tip or a sharp end for inserting the insertable part of the medical device, exemplarily the insertable portion of the medical device, into the body tissue.

The insertion tool comprises a shaft configured for receiving the medical device. The term “shaft” as used herein is a broad term and is to be given its ordinary and customary meaning to a person of ordinary skill in the art and is not to be limited to a special or customized meaning. The term specifically may refer, without limitation, to an elongated element. The shaft may extend along and/or parallel to an axis of the insertion tool. The shaft may be made of one or more of a metallic material such as steel, e.g., stainless steel, a plastic material, or a ceramic material. For example, the shaft may comprise an elongated structure punched from a sheet of metal, e.g., by using at least one punching tool. The shaft may have an open structure. The shaft may have a c-profile, also denoted as U shape, e.g., having a surface of a parabolic cylinder. The c-profile may be generated by bending, e.g., by using at least one bending tool. The punching and bending may be performed in separated processes or in a joint process step, e.g., by using a punch bending tool.

The insertion tool may comprise at least one insertion cannula or at least one insertion needle. The term “insertion cannula” as used herein is a broad term and is to be given its ordinary and customary meaning to a person of ordinary skill in the art and is not to be limited to a special or customized meaning. The term specifically may refer, without limitation, to a hollow needle which may be at least partially slotted such as in U shape, triangular shape, elipsoid shape or round shape. The shaft may comprise a slotted cannula, wherein the shaft is at least partially open, thereby forming an open slot along the full length or at least along a part of a length of the shaft. At least the insertable part of the medical device may be received within the insertion cannula, such as within a lumen of the insertion cannula. The term “insertion needle” as used herein is a broad term and is to be given its ordinary and customary meaning to a person of ordinary skill in the art and is not to be limited to a special or customized meaning. The term specifically may refer, without limitation, to a compact needle, exemplarily without a slot and without any hollow parts. The medical device such as a soft cannula of an infusion set of a patch pump may be received on an outer surface of the insertion needle.

The term “first end” as used herein is a broad term and is to be given its ordinary and customary meaning to a person of ordinary skill in the art and is not to be limited to a special or customized meaning. The term specifically may refer, without limitation, to a part of the shaft configured for puncturing the skin of the subject. The insertable first end may comprise a tip portion. The tip portion may be a pointed or sharp tip taper-shaped tip portion, which is usable for inserting the insertable part of the medical device into the body tissue. During insertion, the insertion tool may perform a puncture motion from a distal to a proximal position, thereby creating an incision in a skin of the subject, e.g., a user or patient, transferring at least the insertable part of the medical device into the body tissue, and, subsequently, a motion into the reverse direction, wherein the insertion tool is pulled out from the body tissue, wherein at least the insertable part of the medical device at least partially remains within the body tissue. During insertion, at least the insertable part of the medical device may fully or partially be surrounded by the insertion tool. After insertion, the insertable part of the medical device may remain in the body tissue of the subject. The insertion tool, however, may be retracted from the body tissue of the subject, e.g., into a housing of the inserter, after inserting the medical device.

The term “second end” as used herein is a broad term and is to be given its ordinary and customary meaning to a person of ordinary skill in the art and is not to be limited to a special or customized meaning. The term specifically may refer, without limitation, to a part of the shaft configured for providing an interface to a further element or device. The further element or device may be a needle head. The needle head may be an element of an insertion system, also denoted as inserter, configured for providing a mechanical interface to further elements of the inserter, e.g., a driving mechanism for performing the puncturing motion and the retraction. The adjustable second end may comprise at least one perforation, e.g., one or two holes, configured for supporting a form-fit connection with a further device. For example, the adjustable second end may be connectable via a form-fit connection with a needle head of the insertion system. For example, the insertion tool may be insertable into needle head. The needle head may comprise a mechanical stop, which defines a depth to which the insertion tool can be inserted into the needle head. The adjustable second end may be configured for providing an abutment element adapted for abutting with the mechanical stop of the needle head. Upon insertion of the insertion tool into the needle head, the insertion tool may be glued into the needle head, e.g., using at least one adhesive. The perforation may be configured for providing a form-fit connection via the adhesive between the insertion tool and the needle head. After curing of the adhesive, e.g., by using UV light, the insertion tool and the needle head may be fixedly connected.

The term “adjustable second end” as used herein is a broad term and is to be given its ordinary and customary meaning to a person of ordinary skill in the art and is not to be limited to a special or customized meaning. The term specifically may refer, without limitation, to the fact that the adjustable second end is configured for providing a possibility of extending or reducing a length of the insertion tool and/or amending a shape of the adjustable second end, in particular after production of the insertion tool via punching. For example, the adjusting of the length of the insertion tool may comprise adapting a length of the insertion tool, e.g., in view of a desired insertion depth into the body tissue of the subject. For example, the insertion tool, e.g., with adjusted length, may have a length from 19 mm to 17 mm.

The shaft, at the adjustable second end, may comprise at least one adjustable portion. The term “adjustable portion” as used herein is a broad term and is to be given its ordinary and customary meaning to a person of ordinary skill in the art and is not to be limited to a special or customized meaning. The term specifically may refer, without limitation, to a part situated at the adjustable second end of the shaft and having an adjustable length and/or shape. The adjustable portion may comprise an initial length. For example, the adjustable portion is configured for being reduced from the initial length to a desired length up to a minimum length. The initial length of the adjustable portion can be reduced by 2 mm, preferably by 3 mm.

For example, the adjustable second end comprises at least one undercut. The term “undercut” as used herein is a broad term and is to be given its ordinary and customary meaning to a person of ordinary skill in the art and is not to be limited to a special or customized meaning. The term specifically may refer, without limitation, to a cutout of the shaft and/or an element connecting the shaft, e.g., the elongated structure, and a needle comb. The undercut may be embodied such that it can provide a form-fit-connection with an adhesive, as described above. The undercut may be configured for providing an abutment element adapted for abutting with a mechanical stop of a further device, e.g., the needle head as described above. The cutout may be rectangular. The cutout may have a size of 0.2×0.3 mm. The undercut may be configured for providing reaching a required pullout force of the needle from the needle head. Therefore, the number of required perforations for the form-fit-connection as described above can be reduced, e.g., to one.

For example, the adjustable second end comprises a plurality of undercuts. The undercuts may form a structured adjustable portion. For example, in a cross sectional, 2D view, the structured adjustable portion may comprise a plurality of arms, e.g., a comb-like structure. The undercuts may be spaced apart from each other, e.g., the structured adjustable portion may comprise every 0.5 mm, preferably every 1 mm, an undercut.

For example, the insertion tool comprises a shaft structure with multiple abutment elements to enable an adjustment of the length of the insertion tool, e.g., via a process step of cutting the insertion tool from a needle comb. The term “needle comb” as used herein is a broad term and is to be given its ordinary and customary meaning to a person of ordinary skill in the art and is not to be limited to a special or customized meaning. The term specifically may refer, without limitation, to a carrier strip comprising a multitude of insertion tools, e.g., three or more insertion tools. The needle comb may be produced in at least one punching step. Each of the insertion tools may be connected with the carrier strip via its adjustable second end. Each of the insertion tools of the needle comb may comprise a structured adjustable portion with a plurality of undercuts forming multiple abutment elements along the shaft. The needle comb may be configured for allowing cutting the insertion tools from the needle comb. Each of the undercuts may comprise at least one cutting area. The term “cutting area” as used herein is a broad term and is to be given its ordinary and customary meaning to a person of ordinary skill in the art and is not to be limited to a special or customized meaning. The term specifically may refer, without limitation, to an area on which a cutting tool can act upon. For example, the insertion tool is produced by punching and/or bending, e.g., as part of a needle comb, wherein the adjustable second end is adjustable by cutting, e.g., at the cutting area provided by the respective undercut. Therefore, the length of the insertion tool can be defined when cutting the insertion tool from the needle comb, e.g., by individualizing the insertion tools. The length of the insertion tool may be adapted by cutting at different locations during the cutting step in the production line, in particular in the assembly line. Therewith a length adaption may be realized very time and cost efficient.

For example, as outlined above, the shaft may comprise an elongated structure punched and bent from a sheet of metal. The structured adjustable portion may be generated by using a punching tool. The generating of the elongated structure and the structured adjustable portion may be performed in separated processes or in a joint process step, e.g., by using a punch bending tool. The individual needles may be generated by cutting, e.g., using a cutting tool, from the needle comb, wherein the length of the adjustable portion is adaptable.

For example, the adjustment of the length may comprise extending the length of the insertion tool. The initial length of the adjustable portion may be a minimal length. The adjustable second end may be attachable with at least one adapter configured for extending the initial length to a predefined length.

For example, the adjustment of the adjustable second end may comprise adjusting the shape of the adjustable second end. For example, the adjustable second end may be produced without any abutments. The adjustment of the adjustable second end may comprise addition of at least one abutment element to the adjustable second end, e.g., using at least one adapter configured for adjusting the shape of the adjustable second end.

The term “adapter” as used herein is a broad term and is to be given its ordinary and customary meaning to a person of ordinary skill in the art and is not to be limited to a special or customized meaning. The term specifically may refer, without limitation, to a device configured for adapting, e.g., extending, the length and/or a shape of one connector to another. For example, the adapter may be configured for extending the minimal length of the adjustable portion. The adjustable second end may be attachable with the adapter for extending the initial length to the predefined length. The adapter may have a first end, e.g., with a hole, to be attached to the insertion tool. The term “attachable” as used herein is a broad term and is to be given its ordinary and customary meaning to a person of ordinary skill in the art and is not to be limited to a special or customized meaning. The term specifically may refer, without limitation, to suitability of an element to be connected with a further element. The adjustable second end may be attachable with the adapter by a form-fit and/or force-fit connection. For example, the adjustable second end may be glued into the adapter, e.g., by using at least one adhesive, such as into the hole of the first end of the adapter.

The adapter may have a predefined height. The height may define the amount of extension of the adjustable portion. For example, the initial length of the adjustable portion can be extended by 0.1 to 3 mm. Additionally or alternatively, the adapter may be configured for providing an abutment element, e.g., a bar, adapted for abutting with a mechanical stop of a further device. The adapter may comprise a second end with an abutment element.

The insertion tool and/or the adapter enable to adjust a length without the necessity of redesigning any tools, in particular, a cutting tool and/or to change the interface with a further device. An adaption of the production can be performed easy and quick. The length of the insertion tool may be adapted by cutting at different locations during the cutting step in the production line, in particular in the assembly line. For example, a production tool such as a needle tool, in particular a punching and/or sheet bending unit, does not need to be adapted. Therewith a length adaption may be realized very time and cost efficient. Thus, the adjustable second end, can allow adapting the abutment for the mechanical stop individually during the production of the insertion tool, in particular of the needle assembly with needle head. Adapting of the production tool, e.g., a cutting tool, and additional qualification can be avoided. A requalification of the punching/sheet metal bending tool (which is situated at the manufacturer of the insertion tool) can be avoided. The punching tool, which is situated in the assembly line, only needs to be adjusted, which is significantly faster. The insertion tool according to this disclosure can allow adjusting the length of the insertion tool at a relatively late stage during production, and in particular without requiring adapting or ordering of new production tools. Therefore, the insertion tool advantageously allows for cutting the insertion tool at different length during the production. Thus, the adjusting of the length can be realized quick and easy in the production line. This can have significant positive impact on costs and the time to market of the product. Moreover, the interfaces to the further elements can be maintained unchanged. A required pullout force of the insertion tool from the needle head can be reached by using the undercut(s).

In a further aspect of this disclosure, a kit is disclosed, comprising an insertion tool for inserting at least one insertable part of at least one medical device into a body tissue according to this disclosure, e.g., as described in a first aspect above or as descried in more detail below, and at least one adapter configured for extending the adjustable second end to a predefined length. For possible definitions and options, reference may be made to the disclosure of the insertion tool according to this disclosure.

The shaft, at the adjustable second end, may comprise at least one adjustable portion. The adjustable portion may comprise an initial length. The initial length may be a minimal length. The adjustable second end may be attachable with the adapter for extending the initial length to the predefined length. The initial length of the adjustable portion can be extended, e.g., by 0.1 to 3 mm. The adapter may be configured for providing an abutment element adapted for abutting with a mechanical stop of a further device.

In a further aspect of this disclosure, an adapter for extending a length of an insertion tool is disclosed. The adapter has a predefined height, a first end adapted to be attached to the insertion tool and a second end with a mechanical stop. The insertion tool may be the insertion tool according to this disclosure, e.g., as described in a first aspect above or as descried in more detail below. For possible definitions and options, reference may be made to the disclosure of the insertion tool according to this disclosure.

In a further aspect of this disclosure, a needle comb is disclosed, comprising a carrier strip with a multitude of insertion tools according to this disclosure, e.g., as described in a first aspect above or as descried in more detail below. For possible definitions and options, reference may be made to the disclosure of the insertion tool according to this disclosure.

In a further aspect of this disclosure, a cutting tool is disclosed for producing an insertion tool according to this disclosure, e.g., as described in a first aspect above or as descried in more detail below. For possible definitions and options, reference may be made to the disclosure of the insertion tool according to this disclosure.

Summarizing and without excluding further possible embodiments, the following embodiments may be envisaged:

Embodiment 1: An insertion tool for inserting at least one insertable part of a medical device into a body tissue of a subject, wherein the insertion tool comprises a shaft configured for receiving the medical device, wherein the shaft comprises an insertable first end and an adjustable second end.

Embodiment 2: The insertion tool according to the preceding embodiment, wherein the shaft, at the adjustable second end, comprises at least one adjustable portion, wherein the adjustable portion comprises an initial length.

Embodiment 3: The insertion tool according to the preceding embodiment, wherein the adjustable portion is configured for being reduced from the initial length to a desired length up to a minimum length.

Embodiment 4: The insertion tool according to the preceding embodiment, wherein the initial length of the adjustable portion can be reduced by 2 mm, preferably by 3 mm.

Embodiment 5: The insertion tool according to any one of the preceding embodiments, wherein the adjustable second end comprises at least one undercut.

Embodiment 6: The insertion tool according to the preceding embodiment, wherein the undercut is configured for providing an abutment element adapted for abutting with a mechanical stop of a further device.

Embodiment 7: The insertion tool according to any one of the two preceding embodiments, wherein the undercuts are spaced apart from each other, wherein the undercuts comprise at least one cutting area.

Embodiment 8: The insertion tool according to any one of the preceding embodiments, wherein the insertion tool is produced by punching and/or bending, wherein the adjustable second end is adjustable by cutting.

Embodiment 9: The insertion tool according to embodiment 2, wherein the initial length is a minimal length, wherein the adjustable second end is attachable with at least one adapter configured for extending the initial length to a predefined length.

Embodiment 10: The insertion tool according to the preceding embodiment, wherein the initial length of the adjustable portion can be extended by 0.1 to 3 mm.

Embodiment 11: The insertion tool according to any one of the preceding embodiments, wherein the adjustable second end comprises at least one perforation configured for supporting a form-fit connection with a further device.

Embodiment 12: The insertion tool according to any one of the preceding embodiments, wherein the adjustable second end is connectable via a form-fit connection with a needle head of an insertion system.

Embodiment 13: The insertion tool according to any one of the preceding embodiments, wherein the insertion tool has a length from 19 mm to 17 mm.

Embodiment 14: The insertion tool according to any one of the preceding embodiments, wherein the shaft comprises a slotted cannula, wherein the shaft is at least partially open, thereby forming an open slot along the full length or at least along a part of a length of the shaft.

Embodiment 15: The insertion tool according to any one of the preceding embodiments, wherein the shaft is made of one or more of a metallic material such as steel, a plastic material, or a ceramic material.

Embodiment 16: The insertion tool according to any one of the preceding embodiments, wherein the insertable first end comprises a tip portion, wherein the tip portion is a pointed or sharp tip taper-shaped tip portion, which is usable for inserting the insertable part of the medical device into the body tissue.

Embodiment 17: The insertion tool according to any one of the preceding embodiments, wherein the medical device is selected from the group consisting of: a biosensor, specifically an analyte sensor for detecting at least one analyte in a bodily fluid; an infusion device and an electrical stimulating device.

Embodiment 18: A kit comprising:
an insertion tool for inserting at least one insertable part of at least one medical device into a body tissue according to any one of embodiments 1, 2, 9 to 17,
at least one adapter configured for extending the adjustable second end to a predefined length.

Embodiment 19: The kit according to the preceding embodiment, wherein the shaft, at the adjustable second end, comprises at least one adjustable portion, wherein the adjustable portion comprises an initial length, wherein the initial length is a minimal length, wherein the adjustable second end is attachable with the adapter for extending the initial length to the predefined length.

Embodiment 20: The kit according to the preceding embodiment, wherein the initial length of the adjustable portion can be extended by 0.1 to 3 mm.

Embodiment 21: The kit according to any one of the two preceding embodiments, wherein the adapter is configured for providing an abutment element adapted for abutting with a mechanical stop of a further device.

Embodiment 22: An adapter for extending a length of an insertion tool, the adapter having a predefined height, a first end adapted to be attached to the insertion tool and a second end with a mechanical stop, wherein the insertion tool is an insertion tool according to any one of embodiments 1 to 17.

Embodiment 23: A needle comb comprising a carrier strip with a multitude of insertion tools according to any one of embodiments 1 to 17.

Embodiment 24: A cutting tool for producing an insertion tool according to any one of the preceding embodiments 1 to 17.

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of this disclosure is not restricted by the preferred embodiments. The embodiments are schematically depicted in the Figures. Therein, identical reference numbers in these Figures refer to identical or functionally comparable elements.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows an embodiment of a needle comb;

FIGS. 2A and 2B show an embodiment of a needle comb and of an adapter; and

FIG. 3 shows an embodiment of a needle comb in a perspective view.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

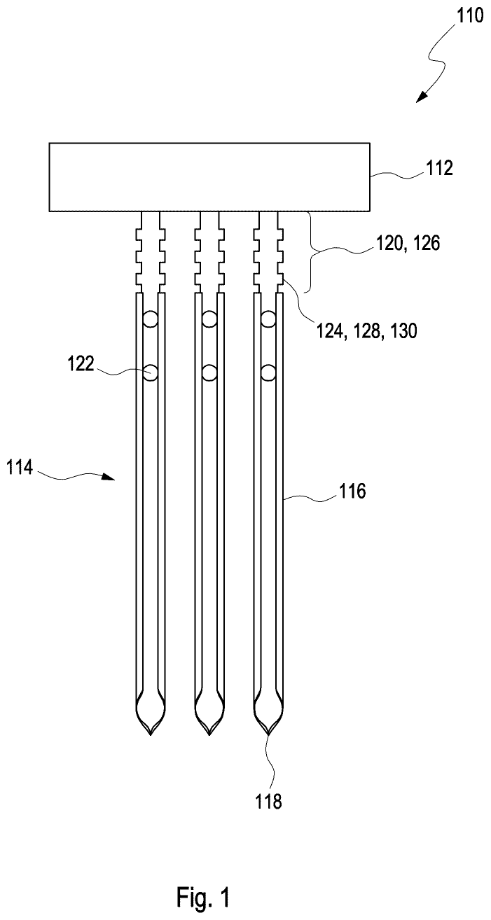

FIG. 1 shows an embodiment of a needle comb 110 comprising a carrier strip 112 with three insertion tools 114 according to this disclosure. Each of the insertion tools 114 is configured for inserting at least an insertable part of a medical device into a body tissue of a subject. The insertion tool 114 comprises a shaft 116 configured for receiving the medical device. The shaft 116 comprises an insertable first end 118 and an adjustable second end 120.

The subject may be a human being or an animal, independent from the fact that the human being or animal, respectively, may be in a healthy condition or may suffer from one or more diseases. The medical device may be configured to be mounted on a skin site, such as a skin site of an extremity of the subject. The extremity may be selected from the group consisting of: an arm, exemplarily an upper arm; a stomach; a shoulder; a back; hip; a leg. Exemplarily, the extremity may be the upper arm. However, also other applications may be feasible. The medical device is selected from the group consisting of: a biosensor, specifically an analyte sensor for detecting at least one analyte in a bodily fluid; an infusion device, e.g., a drug infusion device configured for infusing a liquid drug such as insulin or any other liquid drug into a tissue of the subject; and an electrical stimulating device, e.g., configured for stimulating a body tissue of the subject.

The inserting may comprise an action or process of one or more of transcutaneously or subcutaneously implanting and/or positioning the medical device, exemplarily the insertable portion of the medical device, into the body tissue of the subject. The medical device may partially be inserted into the body tissue. The insertion of the medical device may be performed by using the insertion tool 114. After insertion, at least an insertable part of the medical device may remain in the body tissue of the subject for a predetermined period of time, such as for several hours, exemplarily for one or more days, such as for up to one week, or such as for up to two weeks or even more. The insertion tool 114 may be configured for supporting the insertion of the insertable part of the medical device. The insertion tool 114 may be configured for transcutaneously or subcutaneously inserting at least the insertable part of the medical device into the body tissue, such as by performing an incision or a puncture in a skin of the subject and by transferring the medical device partially into the body tissue. The insertion tool 114 may be removed fully or partially after inserting the medical device at least partially into the body tissue of the subject.

The shaft 116 may be made of one or more of a metallic material such as steel, e.g., stainless steel, a plastic material, or a ceramic material. The insertion tool 116 may comprise at least one insertion cannula or at least one insertion needle. The insertion cannula may be a hollow needle which may be at least partially slotted such as in U shape, triangular shape, elipsoid shape or round shape. The shaft 116 may comprise a slotted cannula, wherein the shaft 116 is at least partially open, thereby forming an open slot along the full length or at least along a part of a length of the shaft 116. At least the insertable part of the medical device may be received within the insertion cannula, such as within a lumen of the insertion cannula.

The insertable first end 118 may be or may comprise a part of the shaft 116 configured for puncturing the skin of the subject. The insertable first end 118 may comprise a tip portion. The tip portion may be a pointed or sharp tip taper-shaped tip portion, which is usable for inserting the insertable part of the medical device into the body tissue. During insertion, the insertion tool 114 may perform a puncture motion from a distal to a proximal position, thereby creating an incision in a skin of the subject, e.g., a user or patient, transferring at least the insertable part of the medical device into the body tissue, and, subsequently, a motion into the reverse direction, wherein the insertion tool 114 is pulled out from the body tissue, wherein at least the insertable part of the medical device at least partially remains within the body tissue. During insertion, at least the insertable part of the medical device may fully or partially be surrounded by the insertion tool 114. After insertion, the insertable part of the medical device may remain in the body tissue of the subject. The insertion tool 114, however, may be retracted from the body tissue of the subject, e.g., into a housing of an inserter, after inserting the medical device.

The adjustable second end 120 may be configured for providing an interface to a further element or device. The further element or device may be a needle head. The needle head may be an element of an insertion system, also denoted as inserter, configured for providing a mechanical interface to further elements of the inserter, e.g., a driving mechanism for performing the puncturing motion and the retraction. The adjustable second end may comprise at least one perforation 122, e.g., one or two holes, configured for supporting a form-fit connection with a further device. For example, the adjustable second end 120 may be connectable via a form-fit connection with a needle head of the insertion system. For example, the insertion tool 114 may be insertable into needle head. The needle head may comprise a mechanical stop, which defines a depth to which the insertion tool can be inserted into the needle head. The adjustable second end 120 may be configured for providing an abutment element 124 adapted for abutting with the mechanical stop of the needle head. Upon insertion of the insertion tool 114 into the needle head, the insertion tool 114 may be glued into the needle head, e.g., using at least one adhesive. The perforation 122 may be configured for providing a form-fit connection via the adhesive between the insertion tool 114 and the needle head. After curing of the adhesive, e.g., by using UV light, the insertion tool 114 and the needle head may be fixedly connected.

The adjustable second end 120 may be configured for providing a possibility of extending or reducing a length of the insertion tool 114 and/or amending a shape of the adjustable second end 120, in particular after production of the insertion tool 114 via punching. For example, the adjusting of the length of the insertion tool 114 may comprise adapting a length of the insertion tool 114, e.g., in view of a desired insertion depth into the body tissue of the subject. For example, the insertion tool 114, e.g., with adjusted length, may have a length from 19 mm to 17 mm.

The shaft 116, at the adjustable second end 120, may comprise at least one adjustable portion 126. The adjustable portion 126 may comprise an initial length. For example, the adjustable portion 126 is configured for being reduced from the initial length to a desired length up to a minimum length. The initial length of the adjustable portion can be reduced by 2 mm, preferably by 3 mm.

For example, the adjustable second end 120 comprises at least one undercut 128. The undercut 128 may be configured for providing an abutment element 124 adapted for abutting with a mechanical stop of a further device, e.g., the needle head as described above. The cutout may be rectangular. The cutout may have a size of 0.2×0.3 mm. The undercut 128 may be configured for providing reaching a required pullout force of the needle from the needle head. Therefore, the number of required perforations 122 for the form-fit-connection as described above can be reduced, e.g., to one.

For example, the adjustable second end 120 comprises a plurality of undercuts 128, e.g., four as shown in FIG. 1. The undercuts 128 may form a structured adjustable portion 130. For example, in a cross sectional, 2D view, the structured adjustable portion 130 may comprise a plurality of arms, e.g., a comb-like structure. The undercuts 128 may be spaced apart from each other, e.g., the structured adjustable portion 130 may comprise every 0.5 mm, preferably every 1 mm an undercut.

For example, the insertion tool 114 comprises a shaft structure with multiple abutment elements 124 to enable an adjustment of the length of the insertion tool 114, e.g., via a process step of cutting the insertion tool from the needle comb 110. The needle comb 110 may be produced in at least one punching step. Each of the insertion tools 114 may be connected with the carrier strip 112 via its adjustable second end 120. Each of the insertion tools 114 of the needle comb 110 may comprise a structured adjustable portion 130 with a plurality of undercuts 128 forming multiple abutment elements 124 along the shaft 116. The needle comb 110 may be configured for allowing cutting the insertion tools 114 from the needle comb 110. Each of the undercuts 128 may comprise at least one cutting area. For example, the insertion tool 114 is produced by punching and/or bending, e.g., as part of a needle comb 110, wherein the adjustable second end 120 is adjustable by cutting, e.g., at the cutting area provided by the respective undercut. Therefore, the length of the insertion tool 114 can be defined when cutting the insertion tool 114 from the needle comb 110, e.g., by individualizing the insertion tools 114. The length of the insertion tool 114 may be adapted by cutting at different locations during the cutting step in the production line, in particular in the assembly line. For example, a needle tool, in particular a punching and/or sheet bending unit, does not need to be adapted. Therewith a length adaption may be realized very time and cost efficient.

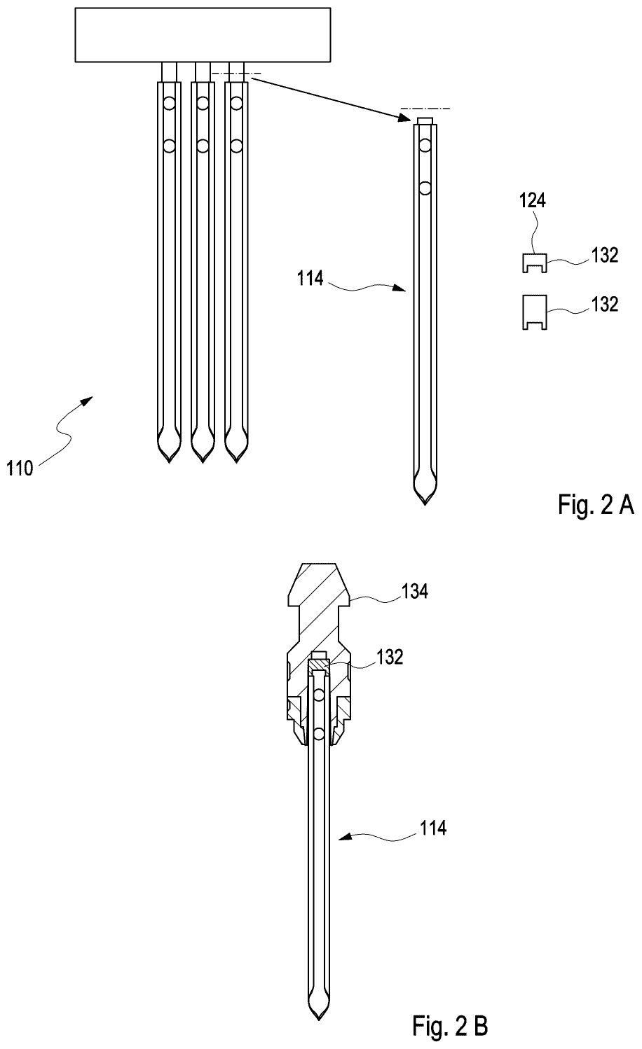

For example, the adjustment of the length may comprise extending the length of the insertion tool 114 as shown in FIGS. 2A and 2B. The initial length of the adjustable portion 126 may be a minimal length. The adjustable second end 120 may be attachable with at least one adapter 132 configured for extending the initial length to a predefined length. FIG. 2A shows an embodiment of a needle comb 110, an individualized insertion tool 114 and two exemplary adapters 132.

For example, the adjustment of the adjustable second end 120 may comprise adjusting a shape of the adjustable second end 120. For example, the adjustable second end 120 may be produced without any abutments, e.g., as shown in FIG. 2A. The adjustment of the adjustable second end 120 may comprise addition of at least one abutment element 124 to the adjustable second end 120, e.g., using the adapter 132 configured for adjusting the shape of the adjustable second end 120.

The adapter 132 may be configured for adapting, e.g., extending, the length and/or a shape of one connector to another. For example, the adapter 132 may be configured for extending the minimal length of the adjustable portion. The adjustable second end 120 may be attachable with the adapter 132 for extending the initial length to the predefined length. The adapter 132 may have a first end, e.g., with a hole, to be attached to the insertion tool 114. The adjustable second end 120 may be attachable with the adapter 132 by a form-fit and/or force-fit connection. For example, the adjustable second end 120 may be glued into the adapter 132, e.g., by using at least one adhesive, such as into the hole of the first end of the adapter 132.

The adapter 132 may have a predefined height. The height may define the amount of extension of the adjustable portion. For example, the initial length of the adjustable portion 126 can be extended by 0.1 to 3 mm. Additionally or alternatively, the adapter 132 may be configured for providing an abutment element 124, e.g., a bar, adapted for abutting with a mechanical stop of a further device. The adapter 132 may comprise a second end with an abutment element 124. FIG. 2B shows an embodiment in which the insertion tool 114 and the adapter 132 are inserted into a needle head 134.

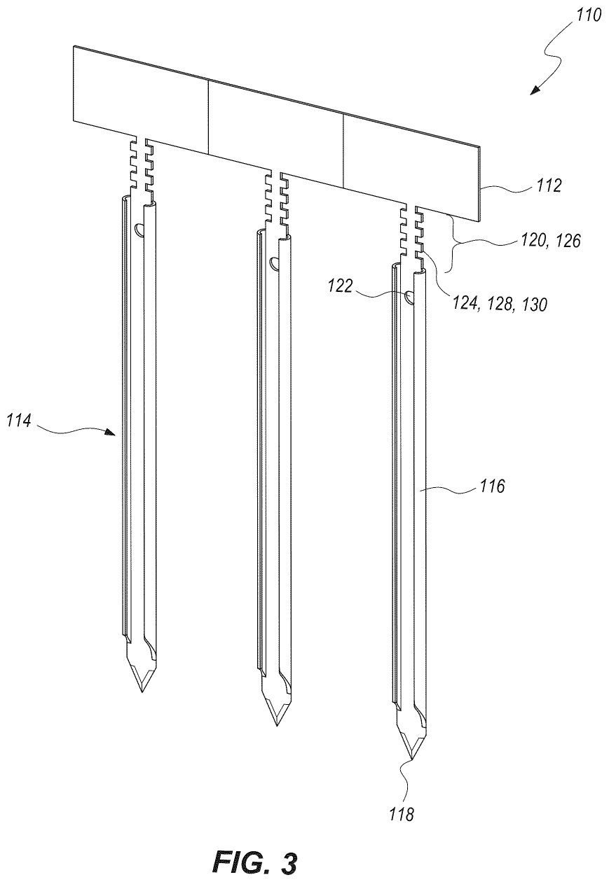

FIG. 3 shows an embodiment of a needle comb 110 in a perspective view. In this embodiment, the needle comb 110 comprises three needles each having a shaft 116. As can be seen in the perspective view, the shaft 116 may have a c-profile. For example, the shaft 116 may have an elongated structure punched and bent from a sheet of metal. Moreover, in this embodiment, each shaft 116 comprises an adjustable second end 120, here with four undercuts 128. The adjustable second end 120 may be generated by using a punching tool. The generating of the elongated structure and the adjustable second end 120 may be performed in separated processes or in a joint process step, e.g., by using a punch bending tool. The individual needles may be generated by cutting, e.g., using a cutting tool, from the needle comb 110, wherein the length of the adjustable portion is adaptable.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 needle comb
112 carrier strip
114 insertion tool
116 shaft
118 insertable first end
120 adjustable second end
122 perforation
124 abutment element
126 adjustable portion
128 undercut
130 structured adjustable portion
132 adapter
134 adapters

What is claimed is:

1. An insertion tool for inserting an insertable part of a medical device into a body tissue of a subject, the insertion tool comprising a shaft configured for receiving the medical device, wherein the shaft comprises an insertable first end and an adjustable second end, wherein the adjustable second end is configured for extending or reducing a length of the insertion tool.

2. The insertion tool according to claim 1, wherein the shaft at the adjustable second end comprises at least one adjustable portion having an initial length.

3. The insertion tool according to claim 2, wherein the initial length is a minimal length, wherein the adjustable second end is attachable with at least one adapter configured for extending the initial length to a predefined length.

4. The insertion tool according to claim 2, wherein the adjustable portion is configured for being reduced from the initial length to a desired length up to a minimum length.

5. The insertion tool according to claim 4, wherein the initial length of the adjustable portion can be reduced by an amount selected from the group consisting of up to 2 mm and up to 3 mm.

6. The insertion tool according to claim 1, wherein the adjustable second end comprises an undercut configured for providing an abutment element adapted for abutting with a mechanical stop of a further device.

7. The insertion tool according to claim 1, wherein the adjustable second end comprises a plurality of undercuts, wherein the undercuts form a structured adjustable portion.

8. The insertion tool according to claim 1, wherein the insertion tool is produced by punching and/or bending and the adjustable second end is adjustable by cutting.

9. The insertion tool according to claim 1, wherein the insertion tool has a length from 19 mm to 17 mm.

10. The insertion tool according to claim 1, wherein the shaft comprises an open slot along the full length or at least along a part of a length of the shaft.

11. The insertion tool according to claim 1, wherein the medical device is selected from the group consisting of: an analyte sensor for detecting at least one analyte in a bodily fluid, an infusion device, and an electrical stimulating device.

12. The insertion tool according to claim 1, further comprising an adapter configured for extending the adjustable second end to a predefined length.

13. The insertion tool of claim 12, wherein the adapter has a predefined height, a first end adapted to be attached to the insertion tool and a second end with a mechanical stop.

14. The insertion tool of claim 13, further comprising a second adapter having a height different from the predefined height of the adapter.

15. A needle comb comprising a carrier strip with a multitude of insertion tools according to claim 1.

16. A cutting tool for producing an insertion tool according to claim 1.
